(19) United States
(12) Patent Application Publication
(10) Pub. No.: US 2020/0054392 A1
(43) Pub. Date: Feb. 20, 2020

(54) Tissue Ablation Apparatus and Method

(71) Applicant: THE WHITELEY CLINIC LIMITED, Guildford (GB)

(72) Inventors: Mark WHITELEY, Guildford (GB); Jaya NEMCHAND, Guildford (GB)

(21) Appl. No.: 16/608,537

(22) PCT Filed: Apr. 27, 2018
(86) PCT No.: PCT/GB2018/051112

§ 371 Date: Oct. 25, 2019

(30) Foreign Application Priority Data
Apr. 28, 2017 (GB) 1706863.6

(51) Int. Cl. A61B 18/14; A61B 18/24

(52) U.S. Cl. CPC A61B 18/1492; A61B 2018/00047; A61B 18/24

(57) ABSTRACT

This specification describes a tissue ablation apparatus comprising a catheter configured to enter an internal region of a subject's body, and a cooling device configured to cool internal tissue to a desired temperature of between −15 degrees C. and +15 degrees C.

### FIELD

The present invention relates to a tissue ablation apparatus and a method of ablating tissue.

### BACKGROUND

There are a number of heat treatments used in curative and aesthetic medicine that can cause damage if the heat spreads to a non-target area. For example, new approaches to curing excess underarm sweating rely on applying microwave radiation to the body. The microwaves are intended to penetrate the skin and cause water in cells beneath the skin to become excited (i.e. heated) and irreparably damaged. While damaging the cells beneath the skin is intentional, it is important that the outer layer of skin is not damaged by the heat. It can be difficult to actively heat one area while preventing the heat spreading to a non-target area.

In another example, in cold cycling treatments such as catheter ablation of heart tissue, the tissue is supplied alternately with extreme cold and heat in order to damage the target tissue. The treatment may be supplied by means of a catheter which is inserted into a vessel and moved through the vessel to the target area. Both the heat and the cold are of such extreme temperatures as to damage the tissue. Similarly to the above example, it may also be difficult to constrain the extreme heat and cooling to a particular area, thus damaging tissue surrounding the target area. This may be a particular problem in the case of treatment of certain body regions, such as the pelvic region. For example, cold-cycling may not be suitable for use in treatment of gonadal veins (the ovarian vein in females, and testicular veins in males), due to the potential for damaging the organs surrounding such veins, such as the ovaries or testicles. Such damage to these organs may cause a reduction in fertility.

Heat treatment for ablation of veins, such as varicose veins, may require injection of local anaesthetic, such as lidocaine for example, around the vein to be treated. This helps prevent causing pain to the tissues surrounding the vein. However, for long veins, several local anaesthetic injections may be required, causing discomfort.

### SUMMARY

According to a first aspect, the specification describes a tissue ablation apparatus comprising a catheter configured to enter an internal region of a subject's body, and a cooling device configured to cool internal tissue to a desired temperature of between −15 degrees C. and +15 degrees C.

Laboratory and clinical research have shown that cooling the body tissue to less than 15° C. reduces the pain felt when the tissue is stimulated by a source of pain.

The cooling device may be a thermoelectric cooling device based on the Peltier effect. Alternatively or additionally, the cooling device may comprise liquid cooling means.

The cooling device may be configured to cool tissue surrounding a tip of the catheter.

The tip may comprise the cooling device.

The cooling device may be configured to cool internal tissue in a first region, and wherein the apparatus configured to ablate internal tissue in a second region within the first region.

The catheter may be a venous ablation catheter.

The tissue ablation apparatus may be configured to emit electromagnetic radiation to ablate internal tissue.

The tissue ablation apparatus may comprise a laser source and an optical fibre to guide laser light and to emit laser light for ablation of internal tissue.

The tissue ablation apparatus may comprise a radiofrequency source to generate radiofrequency radiation for ablation of internal tissue.

The tissue ablation apparatus may further comprise a controller configured to cause the apparatus to initiate ablation after tissue has been cooled to the desired temperature.

According to a second aspect, the specification describes a method of ablating internal tissue, comprising cooling a region of internal tissue to a desired temperature of between −15 degrees C. and +15 degrees C., and ablating tissue within said region.

The method of ablating internal tissue may further comprise determining the desired temperature based on the internal tissue to be treated.

The method of ablating internal tissue may further comprise initiating ablation after the tissue has been cooled to the desired temperature.

All features described herein (including any accompanying claims, abstract and drawings), and/or all of the steps of any method or process so disclosed, may be combined with any of the above aspects in any combination, except combinations where at least some of such features and/or steps are mutually exclusive.

### BRIEF DESCRIPTION OF THE FIGURES

Embodiments of the present invention will now be described, by way of example only, with reference to the accompanying drawings, in which:

FIG. 1a is a schematic illustration of a tissue ablation apparatus;

FIG. 1b is an enlarged view of a portion of the tissue ablation apparatus;

FIG. 2 is a system diagram of the tissue ablation apparatus;

FIG. 3 is an example of a cooling device which may be used in the tissue ablation apparatus;

FIG. 4 is a flow diagram illustrating a method of ablating tissue using the tissue ablation apparatus.

### DETAILED DESCRIPTION

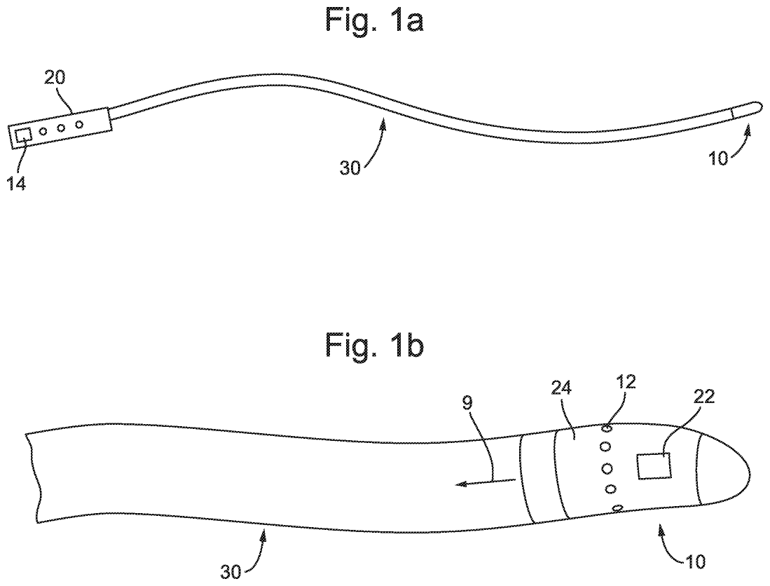

An apparatus 100 for ablation of internal tissue of a subject's body is shown in FIG. 1a. An enlarged view of a portion of the apparatus 100 is depicted in FIG. 1b. The apparatus 100 comprises a cooling device 24, depicted in FIG. 1b, for use in reducing the temperature of internal tissue prior to performing ablation. Ablation is performed by the apparatus 100 on tissue within an area of tissue which has been subject to cooling. For example, in endovenous ablation, a catheter 30 is inserted into a vein, and the cooling device 24 cools a target portion of the vein and the surrounding tissue prior to applying heat to destroy the vein wall.

The cooling device 24 is configured to cool internal tissue to a desired temperature of between −15 degrees C. and +15 degrees C.

Cooling the vein and the surrounding tissue to between −15 degrees C. and +15 degrees C. reduces pain which may be caused by subsequently applied heat. The nerves surrounding the vein are prevented from firing upon cooling, thus relieving the subject from pain during ablation treatment of the vein within the area which has been cooled. Pain relief may therefore be effectively administered via the internal catheter 30. As such, pain relief administered via injection of local anaesthetic to each area of the vein to be treated may not be required, thus improving the comfort of the subject.

Cooling the surrounding tissue to between −15 degrees C. and +15 degrees C. also reduces damage to the surrounding tissue when heat is applied to the vein in the ablation process. Therefore, unnecessary damage to the surrounding tissue may be reduced during ablation. Accordingly, the tissue ablation apparatus 100 described herein may provide protection to tissue which surrounds an area on which the apparatus 100 performs ablation.

As mentioned above, the apparatus 100 comprises a catheter 30 which is configured to enter an internal region of a subject's body. The catheter 30 may be an endovenous ablation catheter. An endovenous ablation catheter 30 is configured to enter a vein of a subject's body and to perform ablation of the vein. That is, the catheter 30 may be dimensioned such that it may be inserted into a subject's vein. In addition, the catheter 30 subjects the vein to heat, causing destruction of the vein wall. In this way, the wall of the vein may be closed.

Temperatures cooler than −15 degrees C. may cause damage, instead of preventing damage. For example, cold cycling ablation uses temperatures below −15 degrees C. to damage the tissue as part of the ablation process. Temperatures higher than +15 degrees C. may not be cool enough to prevent the nerves from firing, and so do not provide pain relief to the subject. In addition, temperatures higher than +15 degrees C. may not be cool enough to protect the tissue surrounding the ablation target region from the heat generated in the ablation process.

It will be understood that the tissue ablation apparatus 100 may be configured to perform ablation on other internal body tissues. Accordingly, the catheter 30 may be configured for insertion into a specific body part. In one example, the catheter 30 may be configured for insertion into the oesophagus of a subject to perform ablation on the internal oesophagus tissue.

When performing endovenous ablation, the cooling of the vein caused by the cooling device may cause the vein to contract. As such, the contact between the catheter 30 and the vein may be increased, thus improving the ablation process.

The catheter 30 may comprise a tip 10, depicted in the enlarged view of FIG. 1b. The cooling device 24 may be configured to cool tissue surrounding the tip 10 of the catheter 30. For example, as shown in FIG. 1, the tip 10 may include the cooling device 24. The cooling device 24 may be provided inside the tip 10 of the catheter 30. Alternatively, the cooling device 24 may form an external portion of the tip 10 of the catheter 30.

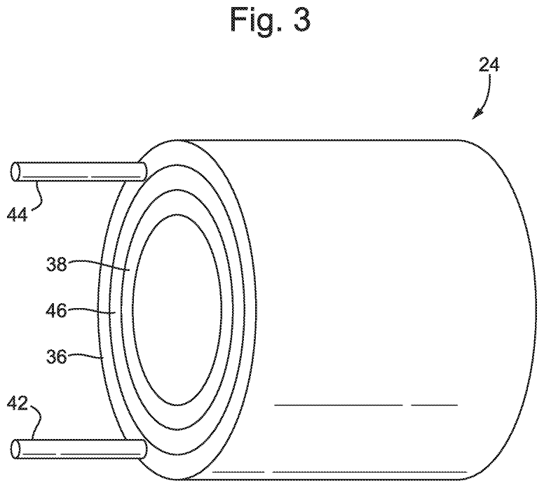

The cooling device 24 may be a thermoelectric cooling device. The thermoelectric electric cooling device 24 may be configured to provide cooling through the Peltier effect, described in more detail with reference to FIG. 3.

In another embodiment, the cooling device 24 may comprise liquid cooling means. For example, cooled liquid may be provided via the catheter 30 to the region to be cooled. The catheter 30 may comprise a portion which conducts heat from the body tissue to the cooled liquid, thus having a cooling effect on the body tissue. The heat conductive portion which forms the cooling device in conjunction with the cooling liquid may be provided in the tip 10. In addition, cooling liquid may be introduced into a catheter 30 which uses a thermoelectric cooling device as a primary cooling source. In this case, the cooling liquid receives heat generated by the thermoelectric cooling device. Cooling liquid may be any liquid approved as a biomaterial. It will be appreciated that the cooling device 24 may comprise any suitable cooling means.

The cooling surface of the cooling device 24 may form part of the outer surface of the tip 10. Alternatively, the tip 10 may comprise a housing, with the cooling device 24 disposed inside the housing.

When the cooling device 24 does not form the outer surface of the tip 10, at least a portion of the tip 10 is fabricated from an expandable material adapted to expand and contract with thermal expansion and/or contraction of the cooling device 24. The material is selected to be highly thermally conductive to maximise thermal energy transfer from the cooling device 24 to the tissue to which it is applied. For example, the tip 10 may include a portion surrounding the cooling device which is fabricated from fine gauge stainless steel, other metals, alloys or a similar non-metal material such that the lower surface is sufficiently durable to withstand sudden temperature changes.

Furthermore, the tip 10 may be cylindrical so that it can easily be inserted into the body. Particularly, a cylindrical shape is useful when the catheter 30 is used to cool the inner surface of a blood vessel or other tubular structure. The cylindrical tip 1o may be a hollow cylinder, i.e. having an opening 18 at its central region along the longitudinal axis of the tip 10. In these embodiments, the cooling device 24 may fully surround the outer surface of the tip 10, while heat generated by the cooling device 24 is dissipated into the tip's 10 central region (i.e. the opening 18). The opening 18 may comprise vents and/or fins for dissipating heat energy away from the tissue, for example to be directed up a hollow portion of the catheter 30 in direction a depicted in FIG. 1b.

The apparatus 100 may be configured to perform ablation by any suitable means. In some examples, the apparatus 100 may be configured to emit electromagnetic radiation to ablate internal tissue. For example, the tip 10 of the catheter 30 may be configured to emit electromagnetic radiation which is absorbed by the surrounding tissue and converted to heat energy, which damages the cells of the tissue. In this way, the cells of a tissue such as vein walls may be destroyed. However, as the tissue surrounding the vein wall may be cooled prior to ablation, the tissue surrounding the vein wall may suffer less damage than if it had not been previously cooled.

For example, the apparatus 100 may comprise a laser source and an optical fibre (not shown) to guide laser light, and to emit laser light for ablation of internal tissue. In some examples, the tip 10 of the catheter 30 may be provided with one or more laser light emission regions 12. For example, the tip 10 of the catheter 30 may be configured such that laser light is emitted in a 360 degree range around the circumference of the catheter 30, as depicted in FIG. 1b. The laser light may be emitted from one or more circumferential rings along the length of the tip 10. Alternatively, the laser light may be emitted in a spiral configuration from the tip 10 of the catheter 30. The optical fibre may provide laser light to the tip 10 of the catheter 30 by passing through the catheter 30 from an external laser source. Any suitable laser source may be used for this purpose.

In another example, the apparatus 100 may comprise a radiofrequency source to generate radiofrequency radiation for ablation of internal tissue. For example, the apparatus 100 may utilise microwave frequencies for ablation of internal tissue. In some examples, the tip 10 of the catheter 30 may be provided with a radiofrequency emission element (not shown). The radiofrequency generation may be driven by an external source connected to the tip 10 of the catheter 30. In the case that the cooling device 24 is a hollow cylindrical cooling device 24, a radiofrequency emission element may be provided inside the hollow portion of the cooling device 24, and may be configured to emit radiofrequency through the cooling device 24 to the surrounding tissue.

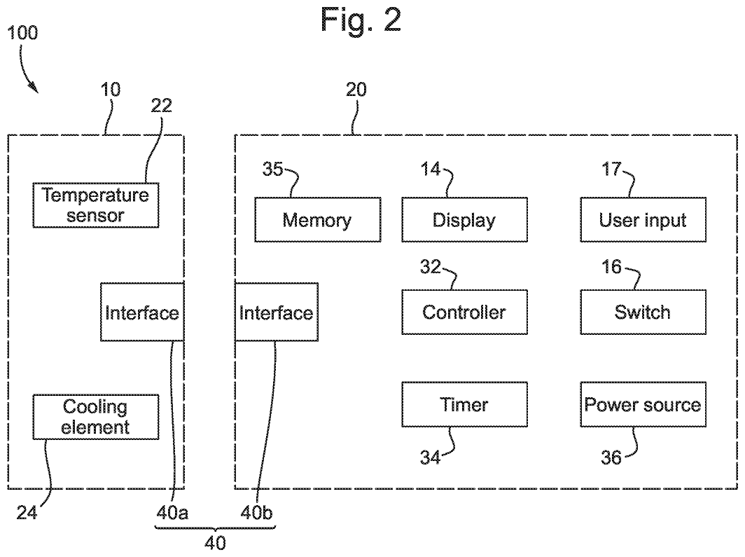

With reference to FIG. 2, the apparatus 100 may comprise a controller 32 for controlling the cooling and ablation operations. For example, the apparatus 100 may comprise control electronics coupled with the cooling device 24 for controlling cooling. In addition, the controller 32 may be configured to initiate ablation. For example, the controller 32 may be configured to turn on a laser source which is configured to transmit laser light through the catheter 30 to the tissue to be ablated. In another example, the controller 32 may be configured to turn on a radiofrequency source which is configured to emit radiofrequency radiation to the tissue to be ablated.

The apparatus 100 may comprise a control unit 20 coupled to the catheter 30. The control unit 20 comprises a controller 32. In the example of FIG. 2, the control unit 20 is a handle. In one example, the handle 20 comprises the controller 32, as depicted for example in FIG. 2.

When working with humans or animals, it is essential to maintain a sterile environment. Particularly, if an instrument is not sterile, the bacteria that may have collected on the instrument can be transferred to a tissue, cause infection. Therefore, medical instruments need to be easy to sterilise, or designed for one-time use. Sterilising instruments typically involves heating the instrument using steam in an autoclave. Clearly, this is not appropriate for instruments having electrical components. Methods of sterilization also include using gamma radiation and Ethylene Oxide (EtO) treatment, but these are costly and labour-intensive.

Therefore, in one embodiment, the present invention provides an apparatus 100 having a disposable catheter 30 and tip 10. This prevents wastage of the control unit 20 which has not come into contact with another human or animal.

The catheter 30, which may be provided separately to the control unit 20, may be encased in a sterile air-tight package prior to use. The cooling device 24 may be disposed in the tip 10.

In some embodiments, the catheter 30 may include a temperature sensor 22 for sensing the temperature of the tissue. The catheter 30 may be provided in the tip 10, for example. The temperature sensor 22 may be any suitable means for detecting the temperature of an object, converting the temperature into temperature information and transmitting the temperature information electronically. For example, the temperature sensor 22 may be a thermocouple. In other embodiments, the temperature sensor 22 may be one of a Negative Temperature Coefficient thermistor, a Resistance Temperature Detector and a semi-conductor-based sensor. The temperature sensor 22 may be formed such that it is in close contact with the subject's tissue when the apparatus 100 is in use. In some examples, the catheter 30 may be provided with more than one temperature sensor 22. For example, a first temperature sensor may be provided in the tip 10, and a second sensor may be provided further up the catheter 30, away from the tip 10. The difference in measured temperature may indicate the cooling rate of the tissue and may indicate how far through the tissue the desired cooling temperature has penetrated. It will be understood that any suitable number of sensors may be used as desired.

In still other embodiments, the temperature sensor 22 may not be necessary. Here, the tissue's initial temperature is assumed to be within a normal range known in the art. For example, the skin's surface temperature on a healthy human is about 34 degrees Celsius. Blood vessels and surrounding tissue close to the skin may be cooler than vessels which are deeper under the skin. This initial temperature, or temperature range from which the most conservative value is chosen for safety, may be received manually from a user input, or received from a storage means. An algorithm executed by a processor or controller in the apparatus 100 is used to determine the length of time and cooling temperature necessary to cool the tissue to a target temperature from the initial temperature. The cooling process is determined to be complete when the determined time expires.

Returning to FIGS. 1a and 2, the control unit 20 may comprise a display 14. The display 14 is, for example, an LCD display. In alternative embodiments, the display 14 may be an LED display or an OLED display. The display 14 shows whether the tissue (for example, a blood vessel and surrounding tissue) is at the appropriate temperature for treatment. In other words, the display 14 provides an indication that the nerves around a target area have been sufficiently cooled such that the target area is numb. The display 14 may display the tissue temperature directly, or a message indicating that the target temperature has been reached. The display 14 may flash to indicate the target temperature has been reached. In some embodiments, the display 14 displays the time left before the tissue will reach the target temperature. The target temperature is typically between −15° C. and +15° C. In some examples, the target temperature is about 0° C. In other words, the target temperature is set to numb nerves, without causing damage to the tissue. Additionally, the target temperature can be set to keep the tissue surrounding the vein wall (or other area to be treated) at a relatively constant temperature while tissue in direct contact with or directly adjacent to the tip 10 of the catheter 30 is heated.

The control unit 20 of the apparatus 100 may also include a sound generator and/or an LED to indicate whether the desired target temperature has been reached.

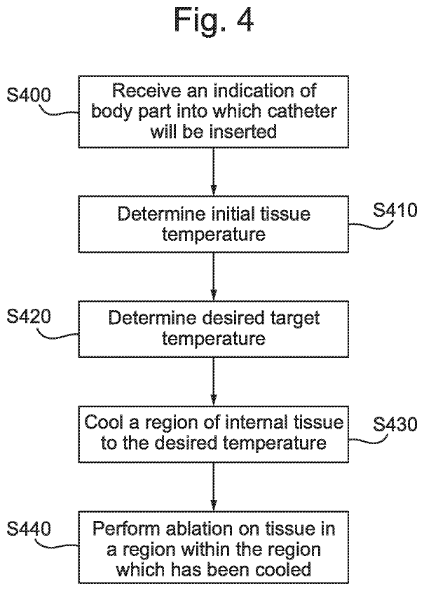

The control unit 20 may include an on/off switch 16 for turning the apparatus 100 on and starting the process described with reference to FIG. 4. The cooling device 24 may deactivate automatically when the target temperature is reached. If the device 100 is left switched on, the cooling device 24 may reactivate when the tissue temperature rises above a threshold temperature greater than the target temperature, in order to keep the temperature of the tissue within a predetermined range.

The control unit 20 may also include a user input 17 for selecting the body part having the tissue which the user intends to cool. For example, the vein wall tissue may react differently to the tissue of the oesophagus, or other internal region of the body. The nerves may be denser, or further away from the region to be treated, and so the cooling device 24 needs to cool for longer. It may be the case that the area of tissue is less sensitive, and so the cooling device 24 can output a lower cooling temperature in order to achieve the target temperature more quickly without causing discomfort. In another example, when performing endoveneous ablation, the tissue in a leg surrounding a thigh vein may have a lower thermal conductivity than the pelvic tissue surrounding the gonadal veins. Therefore, tissue in the pelvic region may require less cooling than the tissue in the pelvic region in order to reach the desired temperature.

The user input 17 shown in FIG. 2 is a manual selector switch. However, in other embodiments, the display 14 is a touchscreen that provides a user input.

In some embodiments, the user input 17 provides a means to manually increase or decrease the temperature of the cooling device 24. The temperature of the cooling device 24 is also known as the cooling temperature.

The apparatus 100 may include an electrical interface 40. The electrical interface 40 may comprise two transceivers 40a, 40b, where one transceiver is disposed in the catheter 30 and the other is disposed in the control unit 20. The electrical interface 30 provides a means for the catheter 30 to transmit temperature information from the temperature sensor 22 to the control unit 20. Moreover, the electrical interface 40 provides a means to transfer power from a power source in the control unit 20 to the cooling device 24 in the tip 10.

The electrical interface 40 may be a wired interface having a plug and socket type configuration. In other embodiments, the electrical interface 30 may be a wireless transmitter, where magnetic coils are used to transfer electrical energy across a gap by inducing current.

Further internal components of the apparatus 100 will now be described with reference to FIG. 2. A controller 32 such as a microprocessor may be used to determine the length of time for which to activate the cooling device 24. This is based on the initial temperature of the tissue and the body part in which the tissue is disposed.

Alternatively, the time is pre-set. The time is set so that the tissue is cooled quickly, but not so quickly as to cause shock. The length of time that the cooling device 24 is used in operation is determined from a timer 34. The timer 34 may be integrated with the controller 32.

The controller 32 may also determine the amount of the current to supply to the cooling device 24 in order for the cooling device 24 to be set at a particular temperature, known as the cooling temperature. The controller 32 can control the current by adjusting a variable resistor or a rheostat disposed between a power source 36 and the cooling device 24. The cooling temperature can be varied while cooling is taking place, until the temperature of the tissue reaches a target temperature. The cooling temperature is optimised based on the cooling time and initial surface temperature such that the tissue is effectively cooled without the patient feeling initial shock as the device 100 makes contact with the tissue.

The memory 35 may store program instructions for allowing the controller 32 to function. For example, the memory 35 may be include a lookup table for associating tissue temperatures with duration of cooling device 24 operation and cooling temperature.

The apparatus 100 may also include a power source 36 for driving the cooling device 24 in the tip 10. The power source 36 may be provided in the control unit 20. The power source 36 according to some embodiments is a battery pack. In other embodiments, the power source 36 is a convertor for converting a mains power supply into a DC voltage for driving the cooling device 24.

FIG. 3 shows a perspective view of a cooling device 24 according to an embodiment. The cooling device 24 may be a thermoelectric device which uses the Peltier effect. The thermoelectric device may be, for example, a thermocycler, single stage, or multistage device. The thermoelectric device is a sandwich structure, with a first conductive material 46 being covered on both sides by a second conductive material 38. A positive electrode 42 and a negative electrode 44 protrude from one of the layers of second material 38 for receiving current from the power source 36 in the control unit 20.

When two conductors are placed in electric contact, electrons flow out of the one in which the electrons are less bound, into the one where the electrons are more bound. The reason for this is a difference in the so-called Fermi level between the two conductors. The Fermi level represents the demarcation in energy within the conduction band of a metal, between the energy levels occupied by electrons and those that are unoccupied.

When two conductors with different Fermi levels make contact, electrons flow from the conductor with the higher level, until the change in electrostatic potential brings the two Fermi levels to the same value. Current passing across the junction results in either a forward or reverse bias, resulting in a temperature gradient. If the temperature of the hotter junction (heat sink) is kept low by removing the generated heat, the temperature of the cold plate can be cooled by tens of degrees.

The first conductive material 40 is an array of N- and P-type semiconductors. The thermoelectric semiconductor material most often used is an alloy of Bismuth Telluride (Bi2Te3) that has been suitably doped to provide individual blocks or elements having distinct “N” and “P” characteristics. Other thermoelectric materials include Lead Telluride (PbTe), Silicon Germanium (SiGe), and Bismuth-Antimony (Bi—Sb) alloys. The second conductive material 38 is for example a copper plate or an aluminium plate. Although not shown, ceramic plates may be disposed on the outside surface of at least one of the second conductive material 38 plates.

FIG. 4 is a flowchart illustrating a method of ablating internal tissue using the apparatus 100.

In step S400, the apparatus 100 may be configured to receive an indication of the body part into which the catheter 30 is to be inserted. For example, the controller 32 may receive “thigh vein” through the user input 17.

In step S410, the temperature sensor may measure the initial tissue temperature, and provides the temperature information corresponding to the measured temperature to the controller 32. Step S410 outlines just one example of receiving an initial surface temperature. In other embodiments, the initial tissue temperature may be assumed using a lookup table. Here, a storage means may store a list of tissues and their corresponding normal temperature. The controller then looks up the necessary tissue or initial temperature when the tissue is input by the user. In still other embodiments, the user can input the initial surface temperature manually using the user input 17.

In step S420, the controller may be configured to determine a desired target temperature based on the body tissue to be treated, the desired temperature being between −15 degrees C. and +15 degrees C. The desired temperature is selected in this range as discussed above to provide the pain relieving effect by numbing nerve endings to provide and the protective effect on the tissue surrounding the ablation target region by, without being so low to cause damage to the tissue. The controller may be configured to determine a cooling temperature of the cooling device 24, which may be colder than or the same as the target temperature. The colder the cooling temperature, the quicker the tissue reaches the desired target temperature. However, when working with humans and animals, it is not desirable to place an item that is too cold on a tissue, or cool the tissue too quickly, as this may shock the patient. Therefore, in some embodiments the controller 32 optimises the cooling time and cooling temperature according to the initial tissue temperature, target temperature and optionally the selected body part. The target temperature is typically between −15 degrees Celsius and +15 degrees Celsius. Preferably, the target temperature is between −5 degrees C. and 0 degrees C.

In step S430, the apparatus 100 is configured to cool a region of internal tissue to the desired temperature of between −15 degrees C. and +15 degrees C. The controller may 32 control the current provided to the cooling device 24 in order to set the cooling temperature if an electric cooling device is utilised. After the calculated time period, the controller 32 may automatically turn off the cooling device 24. In other embodiments, the tissue temperature may be measured at predetermined intervals, such as once per second, and the controller 32 turns off the cooling device 24 when the target temperature is reached. As described above, the tissue temperature may be determined at locations along the length of the catheter 30 other than the tip 10, to determine how far the cooling effect has spread through the surrounding tissue.

In a second step S440, the apparatus 100 is configured to perform ablation on an ablation target region, the ablation target region being located within the region of tissue which has been cooled to the desired temperature.

Advantages of the apparatus 100 of the invention reside in reducing the discomfort caused by ablation of an internal tissue such as, but not limited to, a vein. In addition, the cooling provided by the apparatus 100 helps to protect tissue surrounding the ablation target region from damage caused by the heat from ablation. Furthermore, the cooling contracts the vein to increase the contact area between the ablation target region and the catheter 30 tip 10.

1. A tissue ablation apparatus comprising a catheter configured to enter an internal region of a subject's body, and a cooling device configured to cool internal tissue to a desired temperature of between −15 degrees C. and +15 degrees C.

2. The tissue ablation apparatus of claim 1, wherein the cooling device is a thermoelectric cooling device based on the Peltier effect.

3. The tissue ablation apparatus of claim 1, wherein the cooling device comprises liquid cooling means.

4. The tissue ablation apparatus of any of claim 1, wherein the cooling device is configured to cool tissue surrounding a tip of the catheter.

5. The tissue ablation apparatus of claim 4, wherein the tip includes the cooling device.

6. The tissue ablation apparatus of claim 1, wherein the cooling device is configured to cool internal tissue in a first region, and wherein the apparatus configured to ablate internal tissue in a second region within the first region.

7. The tissue ablation apparatus of claim 1, wherein the catheter is a venous ablation catheter.

8. The tissue ablation apparatus of claim 1, configured to emit electromagnetic radiation to ablate internal tissue.

9. The tissue ablation apparatus of claim 8, comprising a laser source and an optical fibre to guide laser light and to emit laser light for ablation of internal tissue.

10. The tissue ablation apparatus of claim 8, comprising a radiofrequency source to generate radiofrequency radiation for ablation of internal tissue.

11. The tissue ablation apparatus of claim 1, further comprising a controller configured to cause the apparatus to initiate ablation after tissue has been cooled to the desired temperature.

12. A method of ablating internal tissue, comprising cooling a region of internal tissue to a desired temperature of between −15 degrees C. and +15 degrees C., and ablating tissue within said region.

13. The method of ablating internal tissue according to claim 12, further comprising determining the desired temperature based on the internal tissue to be treated.

14. The method of ablating internal tissue according to claim 12, further comprising initiating ablation after the tissue has been cooled to the desired temperature.
